(19) United States
(12) Patent Application Publication
(10) Pub. No.: US 2012/0330335 A1
(43) Pub. Date: Dec. 27, 2012

(54) PERCUSSION TOOL FOR BREACHING INTRAVASCULAR OCCLUSIONS

(75) Inventors: Avraham Shekalim, Nesher (IL); Noam Peleg, Gan Ner (IL)

(73) Assignee: CardioSert Ltd., Yokneam (Moshava) (IL)

(21) Appl. No.: 13/532,839

(22) Filed: Jun. 26, 2012

Related U.S. Application Data
(60) Provisional application No. 61/501,223, filed on Jun. 26, 2011.

(51) Int. Cl. A61B 17/22

(52) U.S. Cl. 606/159

(57) ABSTRACT

A tool for use at an intra-body location during a minimally invasive surgical procedure includes a mechanical energy accumulator associated with first and second bodies. Cyclic relative motion of a pair of linking elements accumulates energy in the mechanical energy accumulator and then suddenly releases the energy to propel the second body through a range of free motion. The tool may be an over-the-wire tool or may be integrated as part of a guidewire. Various embodiments employ a helical spring element or an elastomer block as the accumulator.

This application claims the benefit of Provisional Patent Application No. 61/501,223 filed Jun. 26, 2011.

### FIELD AND BACKGROUND OF THE INVENTION

The present invention relates to tools for use in intravascular procedures and, in particular, it concerns a percussion tool for use in the treatment of chronic total occlusion (CTO) and related conditions in blood vessels.

Chronic total occlusion of a blood vessel is a condition in which there is complete (or near complete) obstruction of that vessel due to the development of an intravascular lesion. Between 10 and 20 percent of patients undergoing percutaneous coronary interventions (PCI) have CTO. Successful opening of CTO lesions improves anginal status, increases exercise capacity, and reduces the need for bypass surgery. However, PCI of cases of CTO have historically posed problems, with lower success rates, higher equipment costs, and a higher restenosis rate.

Conventional intervention tools such as angioplasty balloons are often too flexible or blunt to cross the CTO site, which often contains extremely hard, calcified tissue that may form an impenetrable barrier to the advancement of a guide wire therethrough. Even a less than total occlusion may contain complex structures which may trap or divert the steering end of the guide wire. In view of the great difficulties encountered in attempting to properly position a guide wire across the stenosis, conventional guided atherectomy or dilatation devices such as cutting elements and balloons cannot be used to cross the lesion as long as a guide wire was not inserted through the lesion since they rely on complete wire crossability.

A further problem associated with the use of conventional devices is the risk of perforating the blood vessel being treated. For example, a guide wire or cutting tool, when advanced, may cause dissection of the tissues of the arterial wall instead of the occlusion, thereby creating a false lumen and possibly perforating the artery. If enough blood from a perforated artery accumulates in the pericardial space surrounding the heart, it will result in a condition known as cardiac tamponade in which the heart is compressed and emergency surgical intervention is required to avert heart failure and death.

Another reason that conventional types of apparatus are typically ineffective in treating total or near total occlusions is that conventional balloon catheter shafts and guidewires do not perform well under the compressive loading and torque loading that are required in order to advance such devices across a CTO lesion.

Various approaches have been proposed for breaching such occlusions. Examples include U.S. Pat. No. 5,968,064 and US Patent Application Publication No. 2007/0088380.

It is believed that percussive impact on the occlusion with a blunt tool is particularly effective at breaching the occlusion while minimizing risk of perforating the blood vessel. However, direct delivery of mechanical energy to the handle of a tool has been found to have a low efficacy. This is believed to be due to the inherent flexibility and energy absorbing characteristics of a long catheter following a circuitous path through the blood vessels of the patient to the site of the occlusion.

### SUMMARY OF THE INVENTION

The present invention is a percussion tool for breaching intravascular occlusions.

According to an embodiment of the present invention there is provided, a tool for use at an intra-body location during a minimally invasive surgical procedure, the tool comprising: (a) a first body; (b) a second body; (c) a mechanical energy accumulator associated with the first and second bodies; and (d) a pair of elongated flexible linking elements for actuating the tool at an intra-body location from outside the body, the pair of linking elements being associated with the first body and with at least one of the mechanical energy accumulator and the second body, wherein cyclic relative motion of the pair of linking elements is effective to accumulate energy in the mechanical energy accumulator and then suddenly release the energy to propel the second body through a range of free motion.

According to a further feature of an embodiment of the present invention, the tool is integrated as part of a guidewire.

According to a further feature of an embodiment of the present invention, the second body is deployed as a tip of the guidewire.

According to a further feature of an embodiment of the present invention, the mechanical energy accumulator comprises a helical spring that forms at least part of an external surface of the guidewire.

According to a further feature of an embodiment of the present invention, a distal end of the helical spring is attached to, or integrally formed with, the second body.

According to a further feature of an embodiment of the present invention, a proximal end of the helical spring is attached to, or integrally formed with, the first body.

According to a further feature of an embodiment of the present invention, a first of the pair of elongated flexible linking elements is attached to, or integrally formed with, the first body, and a second of the pair of elongated flexible linking elements is configured to selectively engage the second body for retracting the second body relative to the first body.

According to a further feature of an embodiment of the present invention, an engagement between the second elongated flexible linking element and the second body is configured to be released when an applied retracting force exceeds a given threshold.

According to a further feature of an embodiment of the present invention, the second elongated flexible linking element includes a mechanical stop for limiting the range of free motion of the second body.

According to a further feature of an embodiment of the present invention, a first of the pair of elongated flexible linking elements is implemented as a hollow tube within which passes a second of the pair of elongated flexible linking elements.

According to a further feature of an embodiment of the present invention, the pair of elongated flexible linking elements is implemented as a pair of concentric hollow tubes.

According to a further feature of an embodiment of the present invention, the mechanical energy accumulator is configured to expand radially as it accumulates energy.

According to a further feature of an embodiment of the present invention, the mechanical energy accumulator includes a block of elastomeric material, wherein the accumulation of energy is performed by axial compression of the block of elastomeric material, resulting in radial expansion of the block of elastomeric material.

According to a further feature of an embodiment of the present invention, the block of elastomeric material is generally cylindrical.

According to a further feature of an embodiment of the present invention, the block of elastomeric material is integrally formed with a layer of elastomeric material covering at least one of the first and second bodies.

According to a further feature of an embodiment of the present invention, the block of elastomeric material is integrally formed with a layer of elastomeric material covering a majority of a surface of the tool.

According to a further feature of an embodiment of the present invention, the block of elastomeric material is integrally formed with an inflatable balloon.

### BRIEF DESCRIPTION OF THE DRAWINGS

The invention is herein described, by way of example only, with reference to the accompanying drawings, wherein:

FIG. 1 is a sequence of schematic cut-away views illustrating the operation of a percussion tool, constructed and operative according to an embodiment of the present invention, for breaching an intravascular occlusion;

FIGS. 2A-2C are enlarged schematic cut-away views illustrating various possible forms of a percussive tip for use in the tool of FIG. 1;

FIG. 3 is a sequence of cross-sectional views illustrating an implementation of the tool of FIG. 1 based on an internal helical spring during a cycle of percussive motion;

FIG. 4A is a schematic view of an alternative implementation of the tool of FIG. 1 based on an internal air spring;

FIGS. 4B-4F are enlarged cross-sectional views taken through the tool of FIG. 4A illustrating a cycle of percussive motion;

FIG. 5 is a sequence of views similar to FIG. 3 in a variant implementation integrated at the tip of a guidewire;

FIG. 6 is a schematic illustration of an actuator handle for actuating cyclic motion of the percussive tools of the present invention;

FIG. 7 is a sequence of views illustrating a further variant over-the-wire implementation of a percussion tool according to an embodiment of the present invention;

FIG. 8 is a sequence of views illustrating a device similar to FIG. 7 in a variant implementation integrated at the tip of a guidewire;

FIG. 9 is a schematic illustration of a further implementation of a tool according to an embodiment of the present invention deployed as part of a flexible catheter in an over-the-wire configuration;

FIG. 10 is a sequence of three cross-sectional views taken through the tool of FIG. 9 illustrating three stages during the cycle of operation;

FIG. 11 is a set of three enlarged views of encircled regions of the views of FIG. 10, respectively, in a case of a user-selected maximum-energy percussive impact;

FIG. 12 is a set of three enlarged views of encircled regions of the views of FIG. 10, respectively, in a case of a user-selected intermediate-energy percussive impact;

FIG. 13 is a set of three enlarged views of encircled regions of the views of FIG. 10, respectively, in a case of a user-selected minimum-energy percussive impact;

FIG. 14A is an enlarged view similar to the first view of FIG. 11, and

FIG. 14B is a further enlarged view of the encircled region of FIG. 14A;

FIGS. 15A and 15B are schematic side and cross-sectional views, respectively, taken through a further implementation of a tool according to an embodiment of the present invention in an over-the-wire configuration;

FIG. 15C is an enlarged view of the encircled region of FIG. 15B,

FIG. 16 is a sequence of views similar to FIG. 15B showing the tool in a sequence of stated during a cycle of operation;

FIG. 17 is a sequence of enlarged views corresponding to the encircled regions of the views of FIG. 16, respectively;

FIGS. 18A-18C are views similar to FIGS. 15A-15C for a stand-alone guidewire implementation of a tool according to an embodiment of the present invention;

FIG. 19 is a sequence of cross-sectional views illustrating an implementation of the tool of FIG. 1 based on a block of elastomer material, the tool being shown in sequential states during a cycle of percussive motion;

FIG. 20 is a sequence of schematic cut-away views illustrating the operation of the device of FIG. 19 for breaching an intravascular occlusion; and

FIG. 21 is a sequence of view similar to FIG. 19 for a stand-alone guidewire implementation of a tool according to an embodiment of the present invention.

### DESCRIPTION OF THE PREFERRED EMBODIMENTS

The present invention is a percussion tool for breaching intravascular occlusions, and a corresponding intravascular surgical system and method.

The principles and operation of tools, systems and methods according to the present invention may be better understood with reference to the drawings and the accompanying description.

In general terms, the present invention provides a percussion device for clearing a pathway through occluded or partially occluded blood vessels in which energy is temporarily stored in a spring or other mechanical energy accumulator, at or near the tip of a catheter, and then released to generate a controlled mechanical percussive effect. The percussive effect is generated by sudden release of the locally stored energy so as to impart motion to a weight which has a defined range of free motion. The weight has a blunt end, thereby minimizing the risk of penetrating the wall of blood vessel.

The weight is defined as having a range of “free motion” in the sense that the acceleration of the weight occurs without requiring any corresponding motion of the elongated elements extending along the length of the catheter. The combination of locally stored energy and a weight with a defined range of free motion ensures that the percussive effect is not influenced by the elastic and/or energy absorbing properties, or by the inertial mass, of the elongated elements extending along the catheter. The result is a localized percussive effect delivering a well defined amount of mechanical energy to the occlusion to achieve either immediate or, more typically, incremental erosion of the occlusion.

The mechanical energy accumulator (also referred to as an “energy storage device”) may be any of a number of suitable devices including, but not limited to, a mechanical spring, such as a helical spring or a body of elastomeric material, and an air pressure spring. For simplicity of description, the term “spring” may be used below as a generic term for such devices, without implying limitation to any particular type of mechanical energy accumulator.

Operation of the device is typically by relative motion of two concentric elements (layers, tubes or rods) in a reciprocating motion which typically performs the following functions:
a. clamps the weight;
b. loads the energy storage device;
c. positions an end stop to define the range of motion of the weight; and
d. releases clamping of the weight which is propelled by energy from the energy storage device in free motion towards the occlusion and/or the end stop.

Functions (a), (b) and (c) may be performed in various orders and/or various combinations, or all simultaneously, and are not necessarily all distinct operations. Function (c) in particular may not be a separately identifiable action, for example, being combined with the motion which loads the energy storage device.

According to certain preferred embodiments of the invention, the energy storage device may provide options for defining more than one level of stored energy, corresponding to operator-selected strength of the percussive impact generated. In a simple implementation, this may be implemented by providing more than one clamping position, corresponding to different levels of stored load in a compressed spring.

The aforementioned relative motion of the elongated elements passing along the catheter is typically achieved by automated, semi-automated or manual motion of an actuator mechanism located outside the body, associated with the catheter handle. The actuator mechanism may include various springs and/or manual or electrical actuators. However, it is a particularly preferred feature of certain embodiments of the present invention that forces applied by the actuator mechanism do not play a significant role in the dynamics of the percussive motion of the weight. The proximal mechanism is typically operative to cause the clamping of the weight, loading of the spring and the releasing of the weight, but the motion of the weight once released is essentially a free motion under influence of the spring, generally unaffected by the mechanical properties of the elongated components extending along the catheter.

Embodiments of the present invention may be subdivided into two subsets, according to whether they are used with a conventional guide wire as an “over-the-wire” (OTW) device, or whether they replace the guide wire as a dedicated “impact guide wire”, which may serve as a guide wire for other devices and/or may be integrated with other devices. A few further details relevant particularly to each of these sets of options will now be discussed.

### Option 1: Over the Wire

According to this option, the device is inserted over a standard guide wire and employed to clear a pathway of corresponding diameter around the guide wire. In many cases, occlusion is incomplete, or at least some region of the occlusion is sufficiently soft to allow a standard guide wire to be advanced through the region of occlusion. However, the greatly reduced cross-section in the region of the partial occlusion renders regular balloon techniques ineffective without preparatory opening of a larger passageway through the occlusion. For such applications, an over-the-wire implementation may be preferred.

In this case, the direction of application of percussion is well defined by the path of the guide wire extending through the region of the occlusion, providing an additional safeguard against damage to surrounding tissue. After breaching of the occlusion by the percussive tool of the present invention, the tool can be withdrawn to make way for a separate device, such as a balloon catheter, to be inserted over the guide wire. Alternatively, the percussive tool of the present invention may be integrated with another device, such as a balloon, which is deployed as an outer layer of the combined device.

The over-the-wire option typically preferably employs two concentric tubes deployed over the guide wire in order to generate the required relative motion between the different components of the percussion mechanism independent of the position along the guide wire.

### Option 2: Impact Guide Wire

According to an alternative approach, the percussive device is integrated at the distal end of a guide wire, for facilitating opening a pathway during insertion of the guide wire.

In this case, the actuating structure may employ a solid central core with a single tube around it, or concentric tubes. This allows a particularly compact construction, preferably making the impact mechanism sufficiently narrow to be used with a standard off-the-shelf guide catheter. In certain preferred but non-limiting cases, the overall diameter of the active percussion mechanism is in the range from about 0.5 mm to about 1 mm.

Optionally, the tip of the mechanism is formed with a bendable axial projection which may be pre-flexed to a desired shape to facilitate steering through branched structures. In such cases, the tip (typically integrated with the percussion weight) should be mounted so as to be non-rotatable relative to the main catheter shaft.

The elongated part of the guide wire extending rearward through the catheter may be the same diameter as the percussion mechanism, or in some cases, may have a smaller diameter, for example, conforming to standard guide wire dimensions.

### SPECIFIC NON-LIMITING EXAMPLES

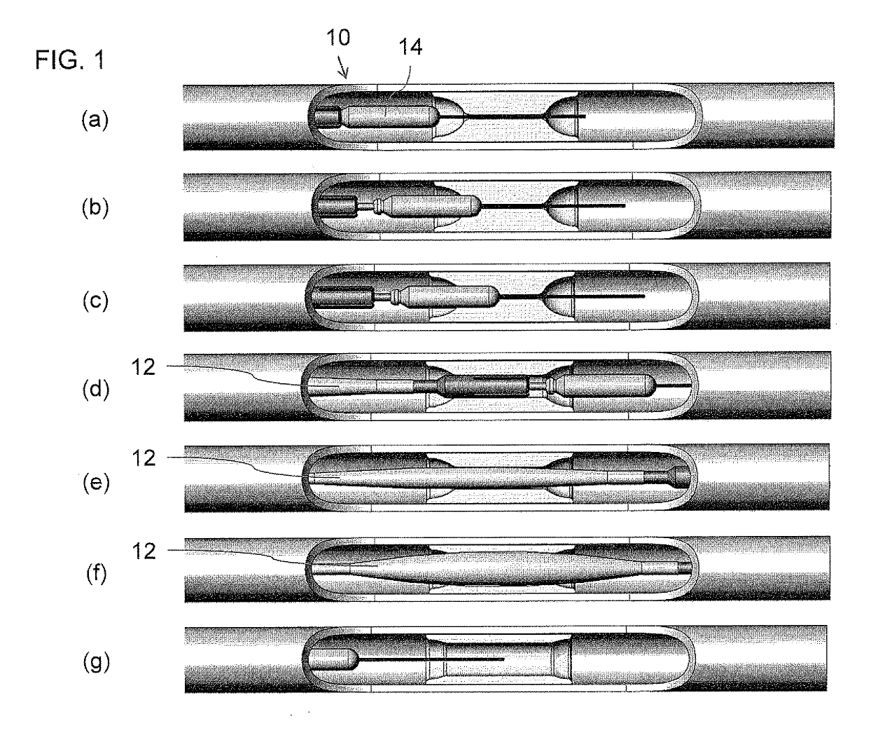

Referring now to the drawings, FIG. 1 shows schematically the sequence of operation of a device constructed and operative according to an embodiment of the present invention, according to which the percussive motion is used repeatedly to drive a channel through an occlusion. In this case, the percussive device 10 is integrated with a balloon device 12. After the percussion device has cleared a pathway through the occlusion (states (a)-(d)), the balloon device is brought into alignment with the remaining part of the obstacle (state (e)) and inflated (state (f) to achieve further broadening of the open channel of the vessel. Clearly, any type of balloon, with or without an associated stent, may be integrated with the percussion device. The entire device is then removed.

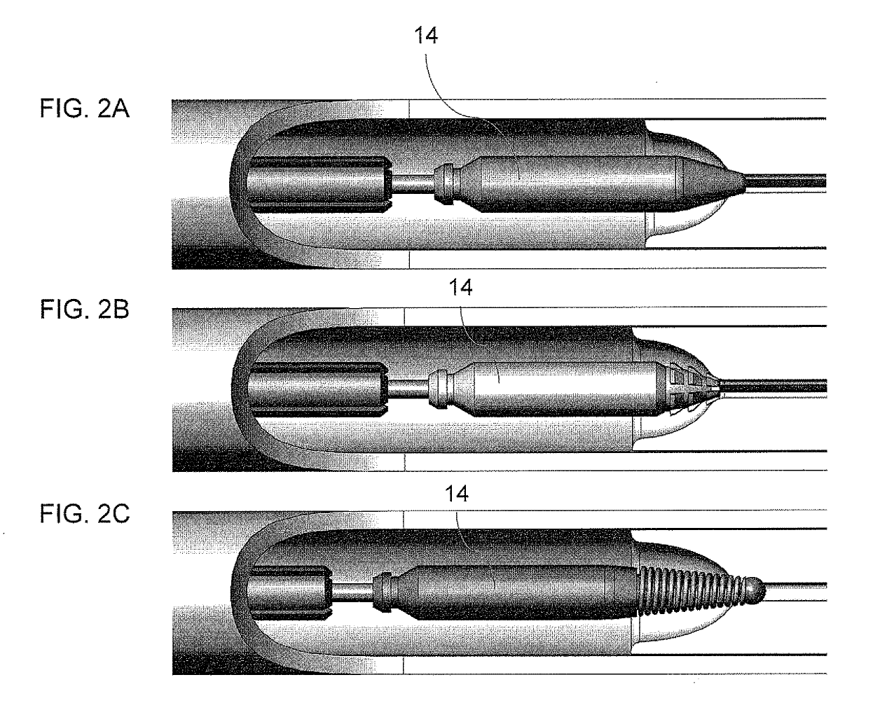

FIGS. 2A-2C illustrate several non-limiting examples of tip configurations for the percussive weight 14 of the present invention. A smooth conical tip (FIG. 2A) tends to achieve outward compression of the displaced material while minimizing the quantity of particulate debris generated. In contrast, the saw-toothed tip structure of FIG. 2B tends to break up the material forming the occlusion more rapidly as it is launched into the occlusion material and then withdrawn prior to the next percussive stroke. A further option illustrated in FIG. 2C is a spring-tipped weight with a rounded end. Particularly for impact guide wire implementations, i.e., without a separate guide wire, a spring extending distally from the weight provides an effective path-seeking tip which tends to follow the path of the blood vessel with minimum likelihood of penetrating through the vessel wall.

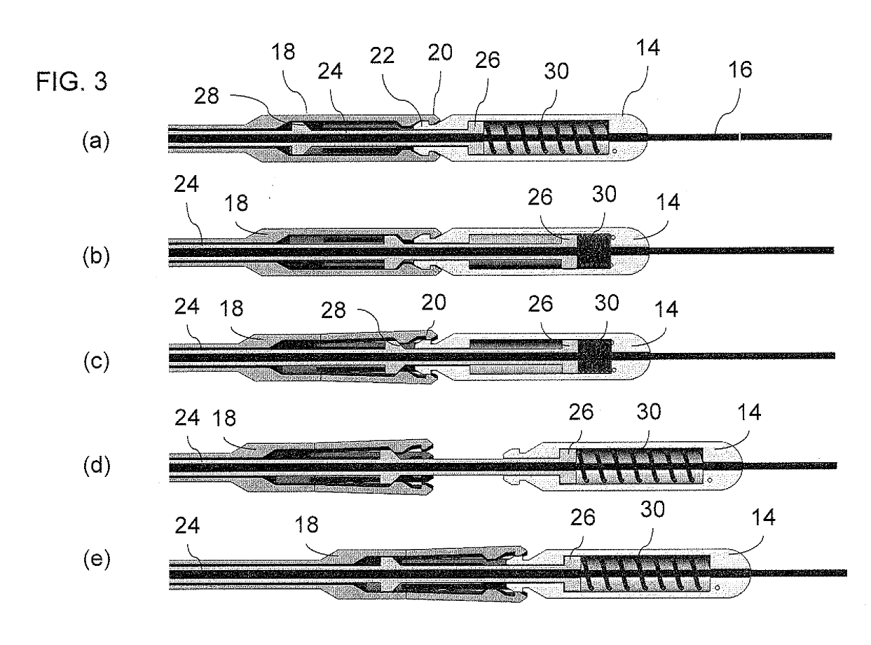

FIG. 3 shows schematically in cross-section an embodiment of the invention for OTW use with a guidewire 16, and it's sequence of cyclic motion. The outer sleeve 18 is first advanced until its clips 20 engage a collar 22 at the base of weight 14, as shown in the state (a). Subsequent advancing of inner sleeve 24 relative to outer sleeve 18 causes a piston 26 within weight 14 to compress the internal helical spring 30, as shown in state (b). At the end of this motion (state (c)), conical wedge surfaces 28 on inner sleeve 24 press outwards on clips 20 to free clamping of the weight. Weight 14 then flies forward freely under action of spring 30 until the rear edge of the internal cavity reaches the rear side of piston 26, which serves also as a stop, as shown in state (d). The outer sleeve is then advanced again relative to the inner sleeve to engage the clips with the collar at the base of the weight to repeat the process.

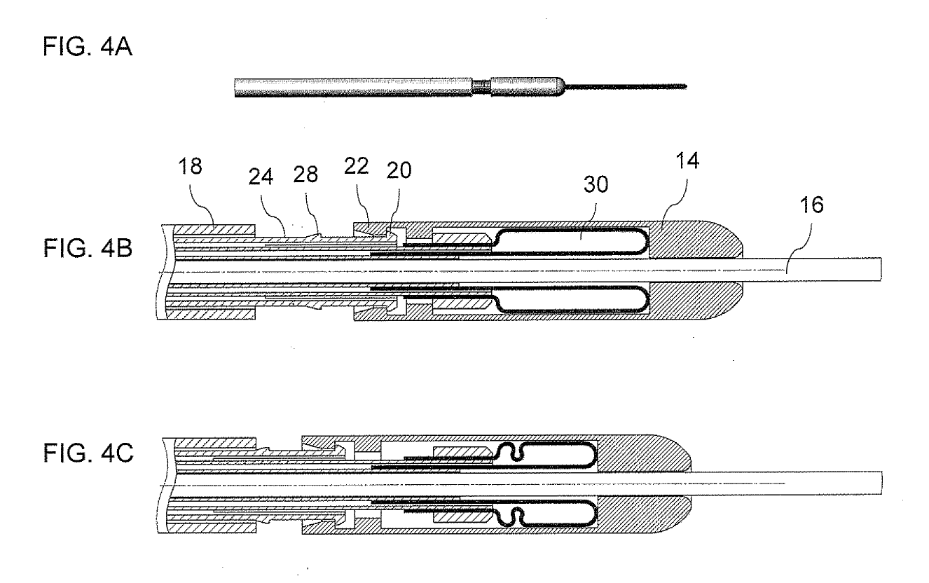

FIGS. 4A-4F show the structure and sequence of operation of an alternative embodiment, similar to that of FIG. 3, but employing an air spring rather than a helical spring. Additionally, the clamping between the weight and the stem of the device is here radially outward, and is released by radially inward pressure applied by the advancing outer sleeve.

The air spring in this case may employ a locally trapped pocket of air, or may be linked to a source of compressed air (or other fluid pressure source) via a channel along the length of the catheter. Even where the pressure is supplied via a channel along the catheter, the air spring acts as a local spring element, with work done on the spring as it is compressed, and recovered from the spring as it is released, and is therefore considered for the purpose of this invention to store energy locally near the tip of the catheter. Parenthetically, for the purpose of the description and claims, the phrase “at or near the tip of the catheter” is used herein to refer to locations within the distal 10% of the catheter's overall length, and most preferably, within the distal 5% of its length.

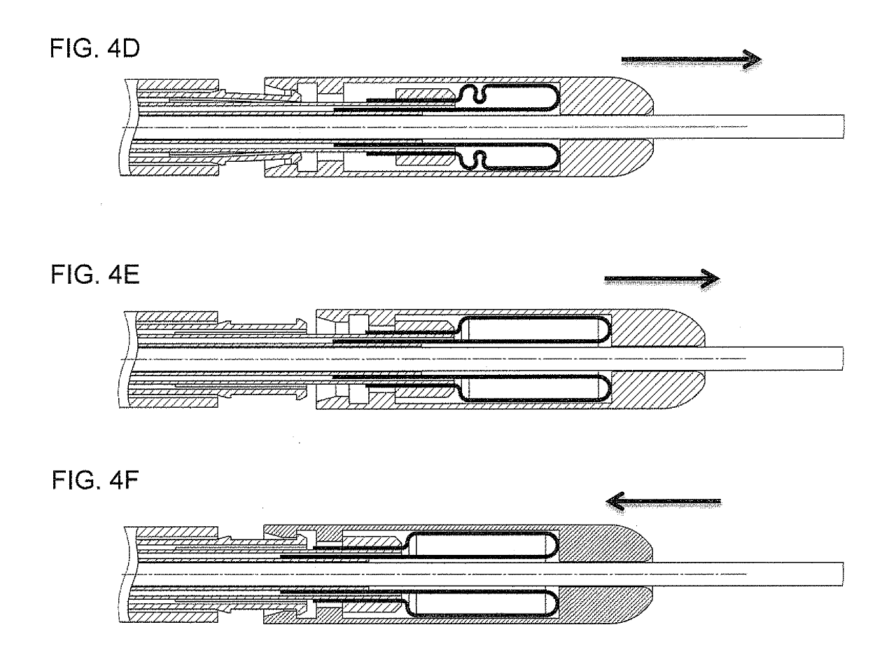

FIG. 5 illustrates the structure and operation of another preferred but non-limiting embodiment of the present invention. This embodiment is structurally and functionally equivalent to that of FIG. 3, but is implemented as an integrated device, integrated with the tip of a guide wire rather than over-the-wire. Equivalent elements are labeled similarly. In this case, the inner element 24 is typically a solid axially extending element.

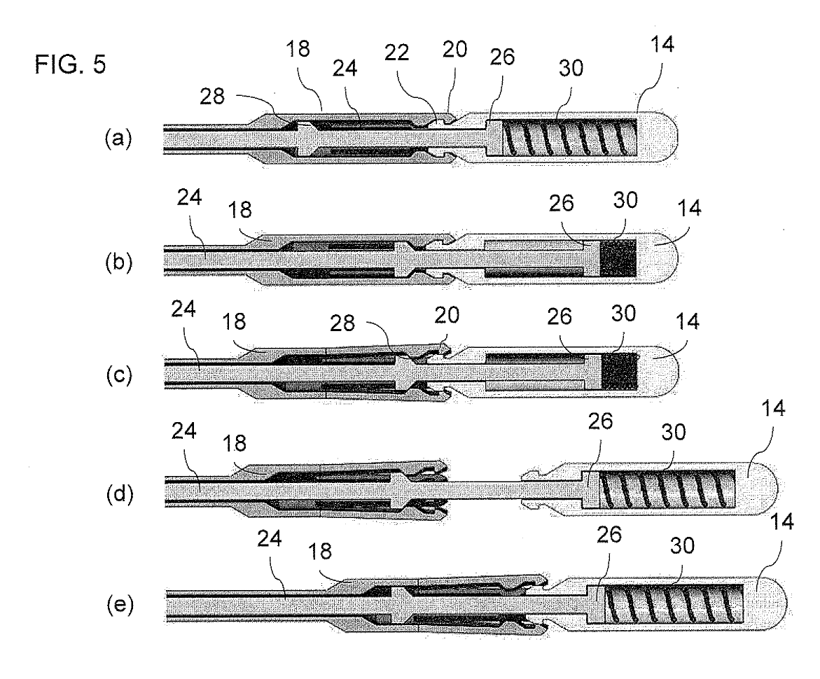

As mentioned earlier, operation of the various embodiments of the present invention is most preferably achieved by generating reciprocating relative motion between an inner and an outer sleeve, or between a central guide wire and a surrounding sleeve, or between any other two coextensive structures corresponding to, or interconnecting with, outer and inner elements of the tool, designated 18 and 24. FIG. 6 illustrates schematically a hand-operated actuation device employing a finger-operated trigger 40 for generating relative displacement in one direction and a return spring for generating the displacement in the other direction. Clearly, the motion may be generated by many other manual or automated mechanisms, as will be clear to one ordinarily skilled in the art.

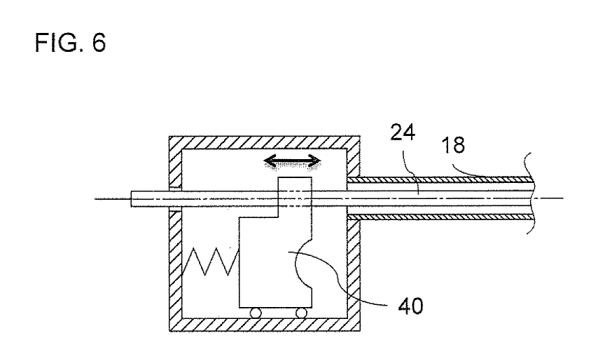

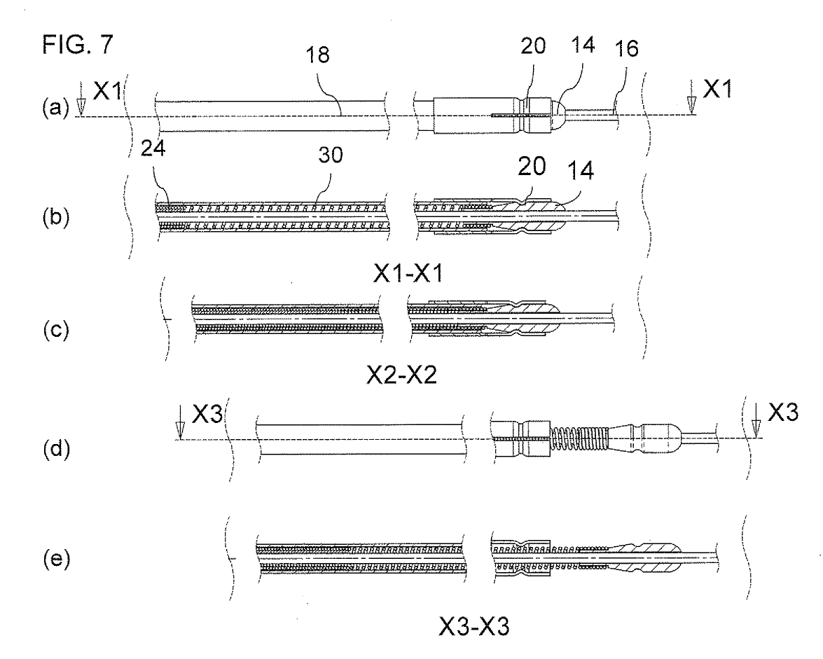

The embodiments illustrated thus far have employed various arrangements for “positive release” of clamping of the weight when the relative motion reaches one extreme of its range of motion. In certain embodiments, however, it is preferred to employ a retention mechanism (clamp) which defines a threshold of force above which the weight is released. FIGS. 7 and 8 illustrate two such examples, in OTW and integrated versions, respectively, in which the weight has a circumferential recess which it clamped by correspondingly shaped resilient sides of an outer housing, connect to the outer sleeve. The inner connection to the sleeve is via a spring element. When the inner connection is withdrawn relative to the outer sleeve, the recess of the weight becomes engaged with the inwardly projecting ridge of the outer housing. When the inner connection is then advanced, compressive force successively builds up in the spring element until it passes the force threshold for displacing the weight from the retention configuration. Optionally, the clamping arrangement and the spring may be designed such that the release force threshold is passed only after windings of the spring have closed against each other in a fully compressed state. The weight then flies forward freely under the action of the spring.

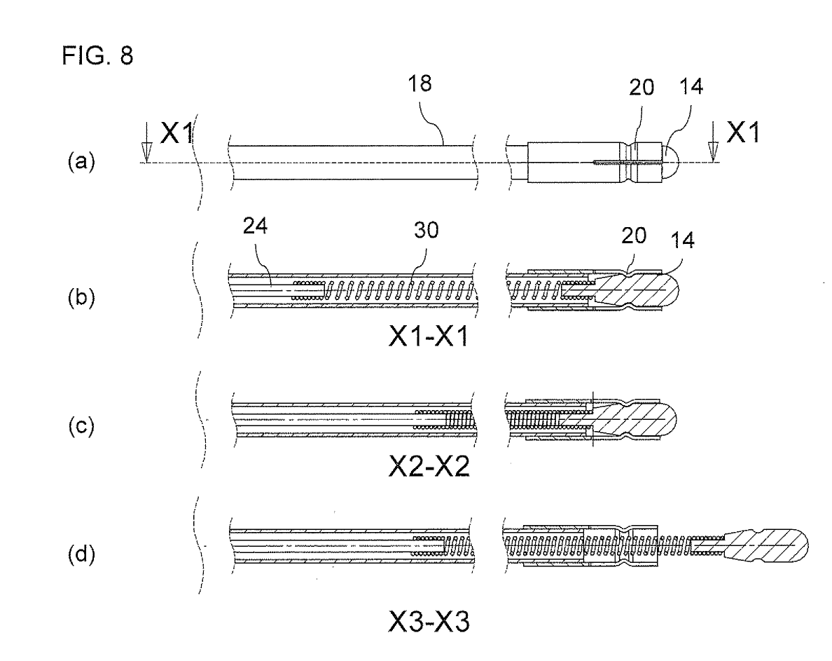

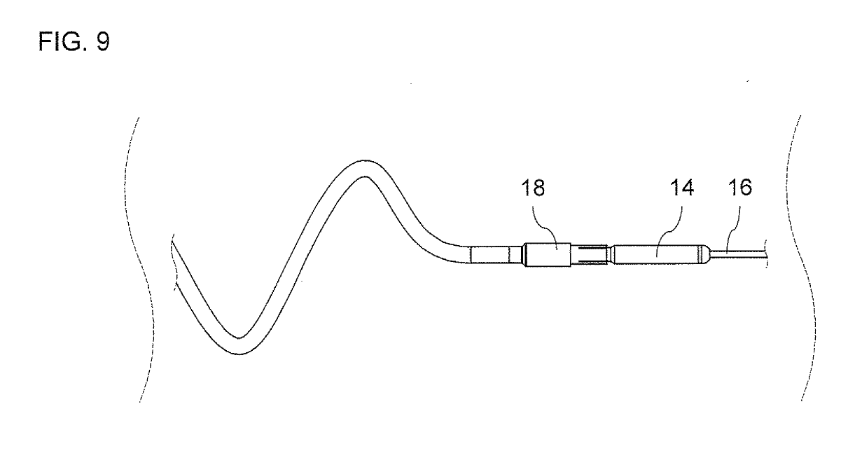

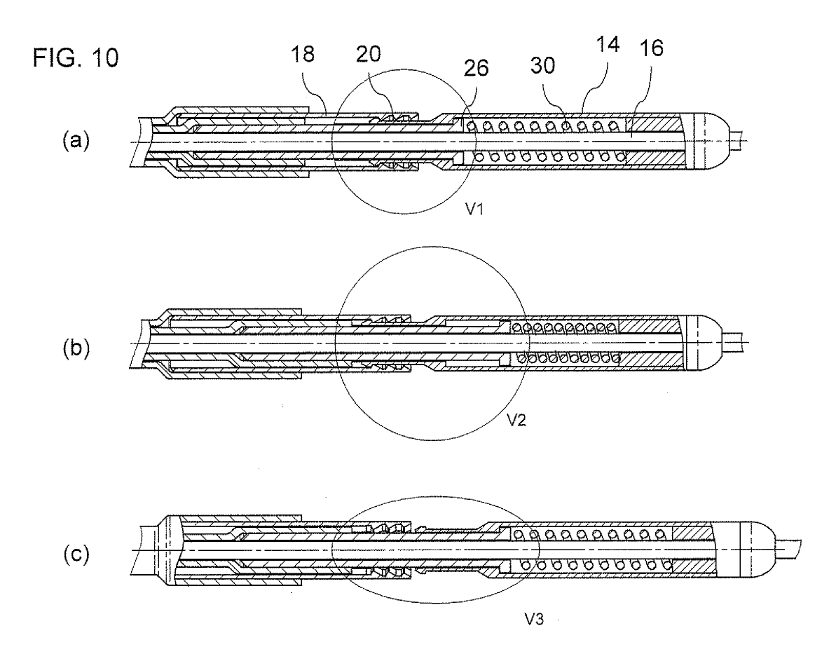

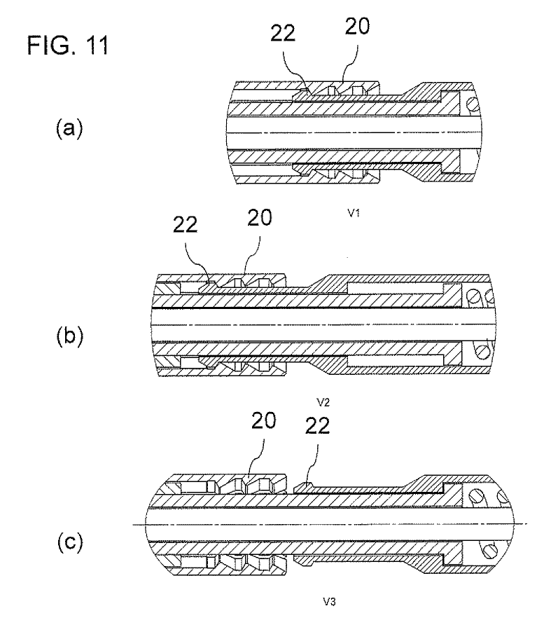

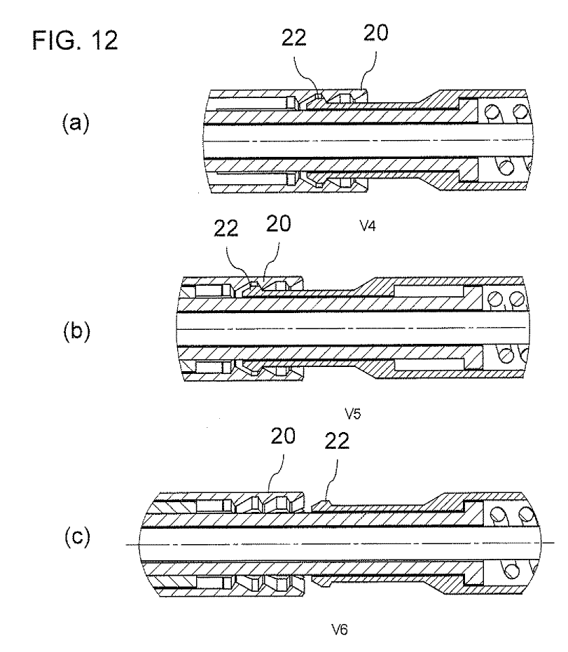

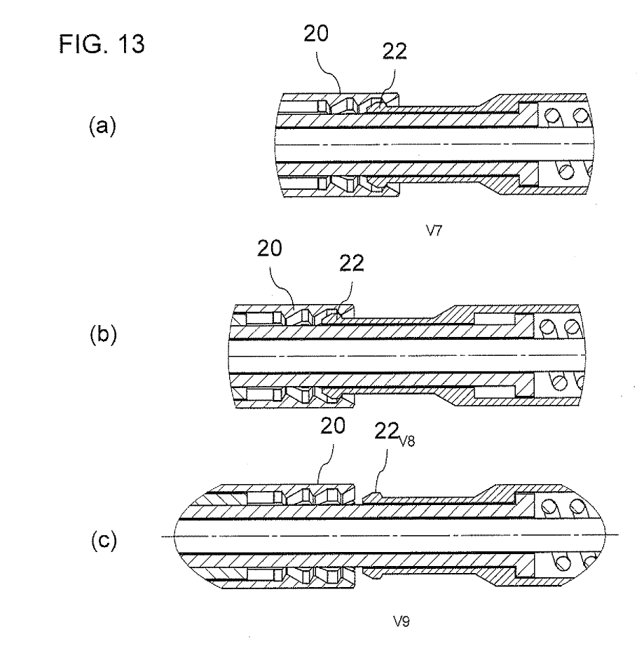

Turning now to FIGS. 9-14B, these illustrate a further embodiment which allows operator selection of one of at least two, and in this case three, different levels of stored energy, allowing the operator to select the magnitude of the percussive impact appropriate for a given procedure, or a given stage of a procedure. In this case, the clamping mechanism defines three distinct clamping positions, which correspond to different levels of stored energy and/or different thresholds for release of the clamping. In this case, there are three distinct clamping positions, each defining a different force threshold for releasing the weight. The different strengths of clamping may result, for example, from the differing distances of the teeth from the root of the slots (visible in FIG. 9) which provide the flexibility of the clamping jaws. FIG. 11 shows the weight engaged at the highest force-threshold position, loaded and then released, while FIG. 12 shows a similar sequence for the medium force-threshold position, and FIG. 13 for the lowest force-threshold position. As best seen in FIG. 14B, according to one preferred implementation, the sequence of teeth may be successively smaller or staggered in order to help ensure that, after clearing one tooth, the weight does not get significantly impeded when passing the subsequent teeth.

Although presented here in the context of a force-threshold released embodiment, it should be noted that the selectable impact force feature may similarly be implemented using a retention configuration with a positive release mechanism. In such cases, the different positions may be designed to correspond to different levels of compression of the spring element prior to release of the clamping mechanism.

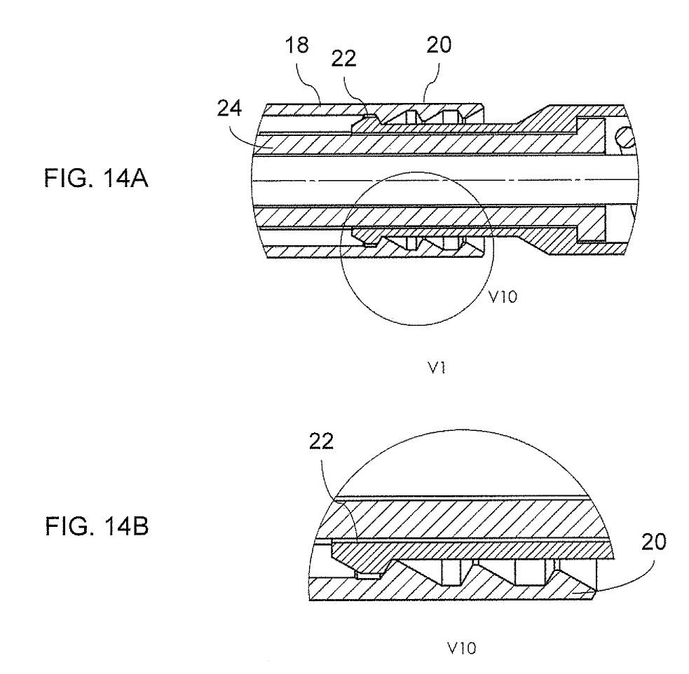

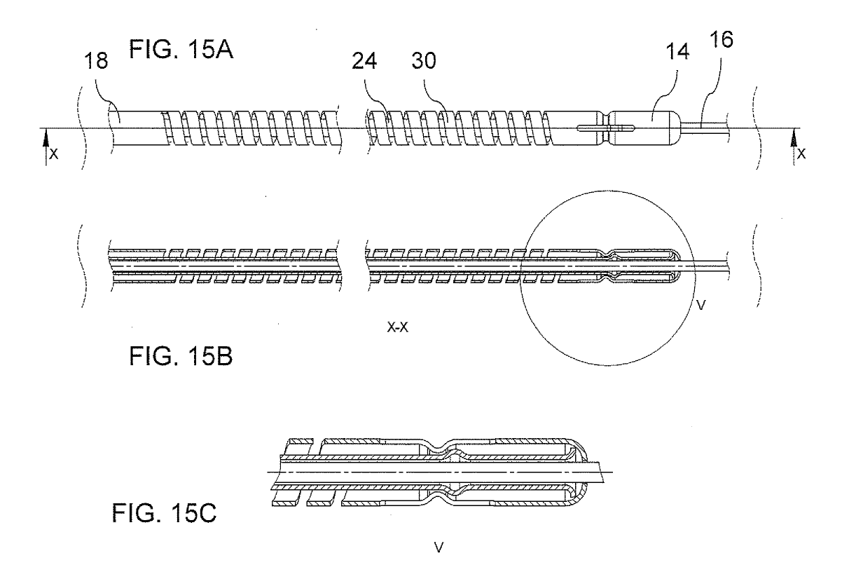

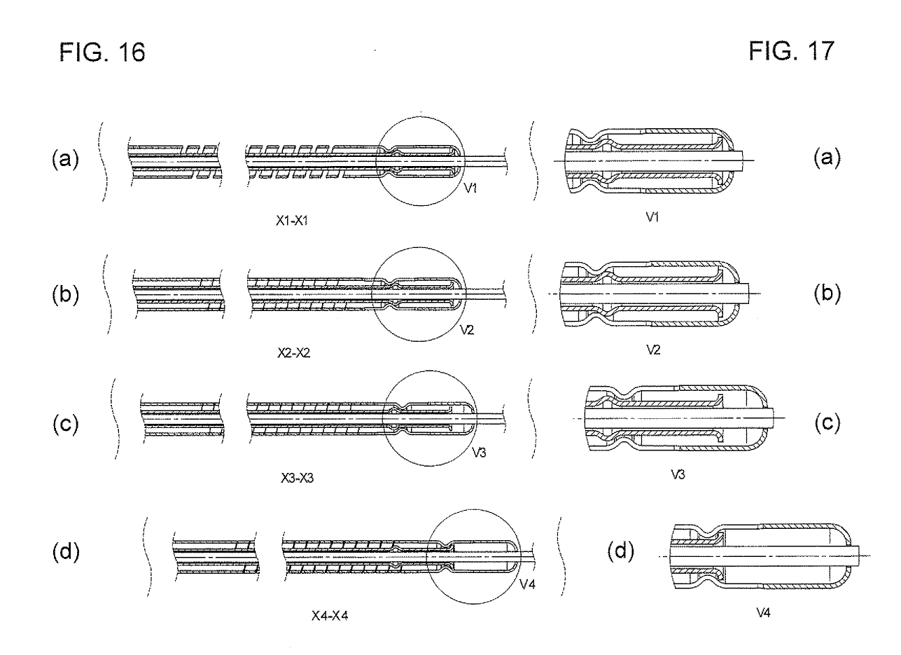

Turning now to FIGS. 15A-17, there is illustrated a further embodiment of the present invention in which the mechanical energy accumulator is formed in the outer sleeve of the structure by providing an elongated helical slot which turns a portion of the outer sleeve/housing into an integrated helical spring configuration. The tip portion of the outer sleeve, distal to the spring configuration, acts as the weight. The tip portion also includes a slotted region with inwardly projecting ridges, which interacts with a radially outward projecting ridge of the inner sleeve to define a retaining mechanism which holds back the tip portion during forward motion of the outer sleeve to compress the spring until the compression reaches a predefined threshold. The threshold may optionally be chosen to be slightly above the force required to fully close the spring, thereby ensuring that the spring is fully compressed before the weight is released. Once the retaining mechanism has been overcome, the weight flies forward freely, under action of the spring, until the inward projection of the tip portion encounters an outwardly flared stop provided at the tip of the inner sleeve, or is otherwise stopped by impact with the occlusion.

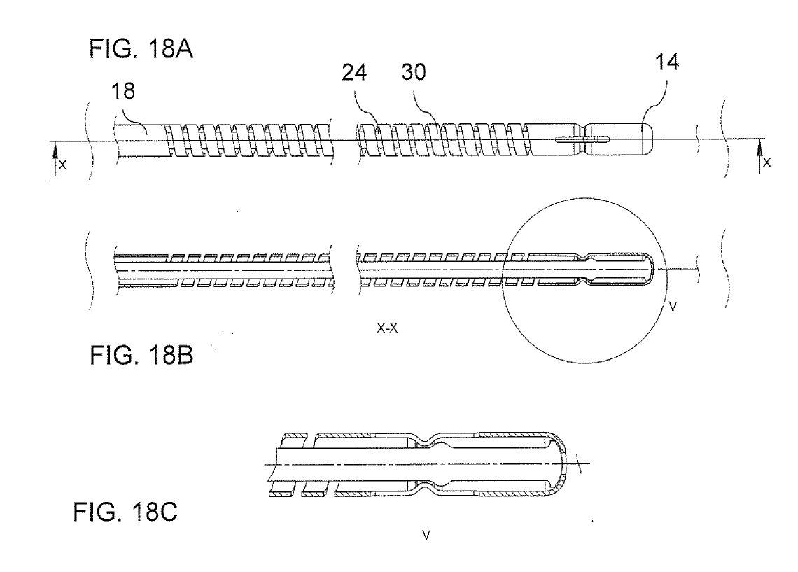

FIGS. 18A-18C illustrate an embodiment similar to that of FIGS. 15A-17 integrated but as a tip-region of a guidewire according to an aspect of the present invention. The structure and function of this embodiment will be fully understood by analogy to the embodiments detailed above.

### Combined Action Expand-and-Advance Embodiments

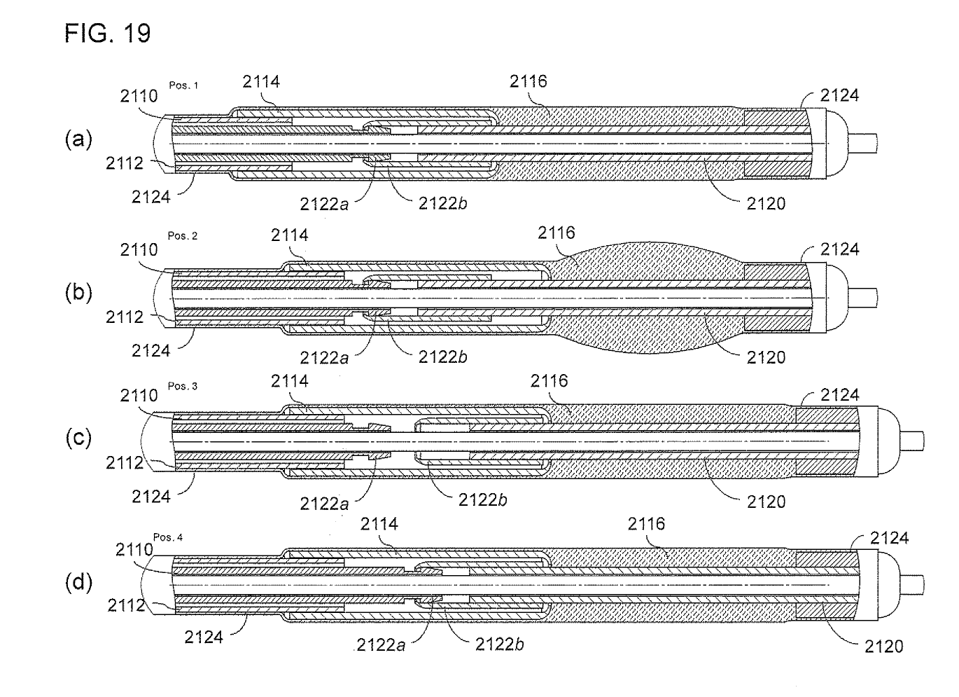

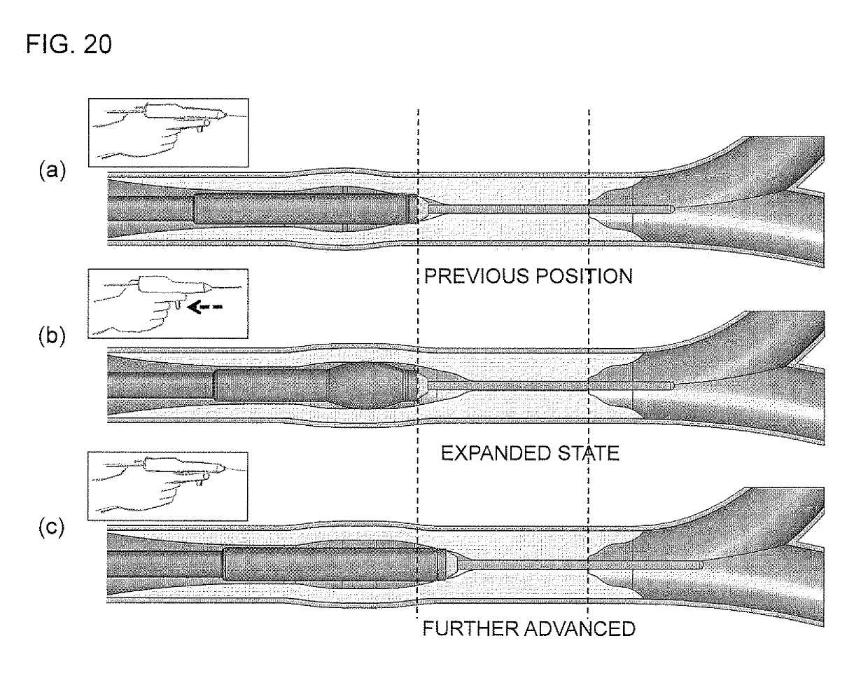

Turning now to FIGS. 19-21, these illustrate further embodiments of the invention. These embodiments introduce an alternative principle of operation according to which radial expansion of a part of the catheter tip helps to open up a passageway for the tip to advance. This radial expansion is followed immediately by advancing of the catheter tip in order to occupy the newly expanded passageway, and the expand-and-advance cycle is repeated. This sequence is illustrated schematically in FIG. 20.

Structurally, a preferred but non-limiting example of the expand-and-advance embodiments may be implemented using a structure very similar to the embodiments described above, where the mechanical energy accumulator is a block of elastomeric material deployed as an outer layer of the device. Due to properties common to most elastomers, axial compression of the elastomeric block during the spring-loading part of the cycle is accompanied by radial expansion, thereby providing the desired outward expanding force. The resulting outward expansion force on the material of the occlusion tends to split apart the material responsible for the blockage, thereby opening up a small passageway ahead of the current position of the device. The subsequent sudden forward release of the stored energy is then effective to rapidly advance the catheter tip further along the split which has been opened, so that the device then repeats the cycle at a position further advanced along the vessel than the preceding cycle. As this sequence continues, the catheter tip advances along the lumen, progressively forcing outwards plaque responsible for the blockage, thereby clearing a passage for insertion of a conventional angioplasty balloon and/or stent, which may be a separate device or may be integrated as part of the device of the invention, all as discussed earlier. The use of radial expansion as the primary mechanism for traversing the occlusion is believed to greatly reduce the risks of distal embolisation and perforation.

Parenthetically, it should be noted that any and all explanations of a physiological mechanism by which the present invention is believed to operate are given merely to facilitate an understanding of the structure and operation of the invention, but that the efficacy of the invention is not dependent upon the accuracy or otherwise of the various theories proposed. In fact, it is to be expected that various additional or alternative mechanisms may come into play in various circumstances such as, for example, where a percussive advancing of the tip of the device is effective to contribute to formation of a crack or other weakening near the axial center of the vessel and the subsequent radial expansion serves both to further open the crack and/or to slightly withdraw the tip of the device from the axial impact zone so that there is sufficient clearance for the tip to accelerate for delivery of an effective percussive impact during the next cycle of operation.

Referring to the structure and operation of the device illustrated in FIG. 19 in more detail, as in previous embodiments, this device is operated by a cyclic relative motion of an outer tube 2110 relative to an inner element 2112. Outer tube 2110 is linked via a connecting sleeve 2114 to the proximal side of the elastomer block 2116, which has a generally cylindrical form. The distal end of elastomer block 2116 abuts a tip block 2118 which is fixed to an inner tip element 2120, which extends proximally through the inside of elastomer block 2116 and beyond. Inner tip element 2120 and the inner element 2112 of the main catheter body terminate in complementary click-engagement features 2122a and 2122b which lock into engagement when pushed together and then release when acted upon by tension above a certain threshold value.

As a result, when outer tube 2110 is advanced relative to inner element 2112 from “Pos. (a)” to “Pos. (b)”, engagement of features 2122a and 2122b prevent tip block 2118 from advancing while connecting sleeve 2114 is pushed forward, thereby axially compressing elastomer block 2116 and causing radial expansion of the block, as shown in Pos. (b). When the compression force exceeds the release threshold of engagement features 2122a and 2122b, inner tip element 2120 breaks free of inner element 2112, allowing elastomer block 2116 to release its stored energy and rapidly advance tip block 2118 to the position of Pos. (c). Inner element 2112 is then advanced relative to outer tube 2110 until features 2122a and 2122b reengage (Pos. (d)), leaving the device ready for the next cycle of expand-and-advance.

A wide range of biocompatible elastomers may be used. By way of one particularly preferred but non-limiting example, silicone rubber may be used. In some cases, it may be advantageous to integrate the elastomer block with an outer layer 2124 covering at least the tip portion of the catheter, and optionally encapsulating most or all of the length of the body of the catheter.

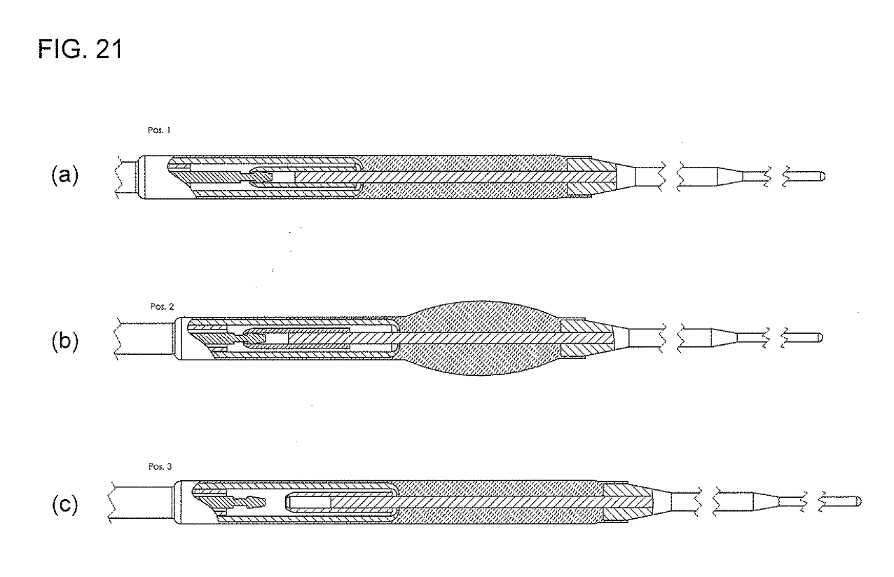

As in all embodiments, these features may be used to advantage in an OTW version as shown in FIGS. 19 and 20, or in an integrated/dedicated structure without reliance on a distally extending guide wire as shown in FIG. 21. The structure and operation of the integrated embodiment of FIG. 21 is fully analogous to that described above with reference to FIG. 19.

It will be appreciated that the above descriptions are intended only to serve as examples, and that many other embodiments are possible within the scope of the present invention as defined in the appended claims.

What is claimed is:

1. A tool for use at an intra-body location during a minimally invasive surgical procedure, the tool comprising:
(a) a first body;
(b) a second body;
(c) a mechanical energy accumulator associated with said first and second bodies; and
(d) a pair of elongated flexible linking elements for actuating the tool at an intra-body location from outside the body, said pair of linking elements being associated with said first body and with at least one of said mechanical energy accumulator and said second body,
wherein cyclic relative motion of said pair of linking elements is effective to accumulate energy in said mechanical energy accumulator and then suddenly release said energy to propel said second body through a range of free motion.

2. The tool of claim 1, wherein the tool is integrated as part of a guidewire.

3. The tool of claim 2, wherein said second body is deployed as a tip of the guidewire.

4. The tool of claim 3, wherein said mechanical energy accumulator comprises a helical spring.

5. The tool of claim 4, wherein said helical spring forms at least part of an external surface of the guidewire.

6. The tool of claim 4, wherein a distal end of said helical spring is attached to, or integrally formed with, said second body.

7. The tool of claim 1, wherein said mechanical energy accumulator comprises a helical spring.

8. The tool of claim 7, wherein said helical spring forms at least part of an external surface of said tool.

9. The tool of claim 7, wherein a distal end of said helical spring is attached to, or integrally formed with, said second body.

10. The tool of claim 9, wherein a proximal end of said helical spring is attached to, or integrally formed with, said first body.

11. The tool of claim 10, wherein a first of said pair of elongated flexible linking elements is attached to, or integrally formed with, said first body, and a second of said pair of elongated flexible linking elements is configured to selectively engage said second body for retracting said second body relative to said first body.

12. The tool of claim 11, wherein an engagement between said second elongated flexible linking element and said second body is configured to be released when an applied retracting force exceeds a given threshold.

13. The tool of claim 11, wherein said second elongated flexible linking element includes a mechanical stop for limiting said range of free motion of said second body.

14. The tool of claim 1, wherein a first of said pair of elongated flexible linking elements is implemented as a hollow tube within which passes a second of said pair of elongated flexible linking elements.

15. The tool of claim 1, wherein said pair of elongated flexible linking elements is implemented as a pair of concentric hollow tubes.

16. The tool of claim 1, wherein said mechanical energy accumulator is configured to expand radially as it accumulates energy.

17. The tool of claim 16, wherein said mechanical energy accumulator includes a block of elastomeric material, wherein said accumulation of energy is performed by axial compression of said block of elastomeric material, resulting in radial expansion of said block of elastomeric material.

18. The tool of claim 17, wherein said block of elastomeric material is generally cylindrical.

19. The tool of claim 18, wherein said block of elastomeric material is integrally formed with a layer of elastomeric material covering at least one of said first and second bodies.

20. The tool of claim 18, wherein said block of elastomeric material is integrally formed with a layer of elastomeric material covering a majority of a surface of the tool.

21. The tool of claim 18, wherein said block of elastomeric material is integrally formed with an inflatable balloon.
